(19) United States
(12) Patent Application Publication
(10) Pub. No.: US 2010/0161016 A1
(43) Pub. Date: Jun. 24, 2010

(54) APPARATUS AND METHOD FOR EFFECTIVELY WARMING A PATIENT

(75) Inventors: Scott D. Augustine, Bloomington, MN (US); Keith J. Leland, Medina, MN (US); Randall C. Arnold, Minnetonka, MN (US); Ryan S. Augustine, Minneapolis, MN (US); Gordon D. Lawrence, Minneapolis, MN (US); Thomas F. Neils, Minneapolis, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: Augustine BioMedical and Design, LLC, Eden Prairie, MN (US)

(21) Appl. No.: 12/339,854

(22) Filed: Dec. 19, 2008

(51) Int. Cl. A61F 7/00

(52) U.S. Cl. 607/112

(57) ABSTRACT

A patient warming blanket includes a pouch and a flexible heating assembly. The flexible heating assembly is enclosed within the pouch and includes, at least in part, a flexible sheet-like heater and a temperature sensor assembly attached to the heater in proximity to a surface thereof; a visible marker is located on an exterior of the pouch to identify a location of the temperature sensor. A method for covering a patient with the patient warming blanket, in order to effectively warm the patient, includes viewing the marker so as to position an area of the blanket, which corresponds to the sensor location, for direct contact with the patient, when the blanket is placed over the patient.

### TECHNICAL FIELD

The present invention pertains to warming a patient, for example, during a surgical procedure, and more particularly to apparatus and methods for effectively warming the patient.

### BACKGROUND

Actively warming a patient, for example, during surgery, with an electric heating blanket can be challenging. Heat transfer between the heating blanket and the patient's body is dependent upon many factors including, for example: a size of a surface area of the heater which is in contact with the patient, a quality of the contact, and an evenness of the heat distributed across the contacting surface area.

An additional factor affecting the amount of heat transfer is a temperature difference (ΔT) between the heating blanket and the patient's skin. The blanket temperature must be greater than the patient's skin temperature, in order to actively transfer heat to the patient, and the greater the blanket temperature, with respect to the patient's skin temperature, the greater the heat transfer. One of the challenges in designing an effective patient warming blanket is in avoiding thermal damage to the patient's skin—there is a relatively narrow temperature range within which to warm the patient without causing thermal damage to the patient's skin.

Normal core temperature of the human body and the temperature of the skin over the core is approximately 37° C. Research has shown that prolonged skin temperatures greater than 43° C. may result in thermal damage, and the greater the temperature, the shorter the exposure time before burns occur. Thus an effective and safe temperature range for warming blankets is between 37° C. and 43° C., wherein a sub-range toward the high end of this range may be necessary for maximal warming effectiveness. Therefore, accurate control of warming blanket temperature, within the safe and effective range, is highly desirable to maximize warming effectiveness.

Experience has shown that electric heaters of patient warming blankets, which have a relatively low watt-density across their surface, are safer than their relatively high watt-density, high powered counterparts, and may consume less electric power. At a constant electrical power input, a low watt-density sheet-like heater, having uniform heat production across its surface, will cool in those areas that make contact with a cooler human body surface. In other words, the relatively cool body surface of the patient acts as a heat-sink which pulls heat out of the sheet-like heater and causes the heater to cool in the areas of contact. As the heater cools, the ΔT between the heater and the body surface gets smaller and the heat transfer decreases. To maintain a desired heater set-point temperature, for those areas of the heater that contact the patient, a controlling temperature sensor of the heating blanket must be located in a patient contact area of the heater, in order to detect the cooling of the heater caused by the heat loss to the patient. As the temperature sensor detects cooling of the heater, in the area of contact, the sensor causes the temperature controller to supply more electric power to the heater in order to increase the blanket temperature, in the area of contact, back up to the desired set-point temperature.

### BRIEF SUMMARY

A patient warming blanket, according to some embodiments of the present invention, includes a flexible sheet-like heater and a temperature sensor assembly, both enclosed within a pouch, which pouch has an exterior surface on which a visible marker is located to identify a location of the temperature sensor assembly. The flexible sheet-like heater is preferably formed from an electrically conductive fabric. The temperature sensor assembly preferably includes a primary temperature sensor and a secondary temperature sensor mounted on a heat spreader. According to some methods of the present invention, by viewing the marker, an area of the warming blanket, which corresponds to the temperature sensor assembly location, is located to directly contact the patient, when the warming blanket is placed over the patient.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1A is a top plan view, including a cut-away section, of a warming blanket, which shows a construction that may be employed by embodiments of the present invention.

FIG. 1B is a plan view of an exemplary patient warming blanket, according to a prior art embodiment.

FIG. 2A is a top plan view of the patient warming blanket shown in FIG. 1A, according to some embodiments of the present invention.

FIG. 2B is a bottom plan view of the warming blanket, according to some embodiments.

FIGS. 3A-E are schematics showing alternative visible markers, which may be employed by embodiments of the present invention.

FIG. 4 is a top plan view of the warming blanket covering a patient, having been positioned according to some methods of the present invention.

FIG. 5 is a top plan view of the warming blanket shown in FIG. 1A, according to some alternate embodiments of the present invention.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention. Examples of constructions, materials, dimensions, and manufacturing processes are provided for selected elements, and all other elements employ that which is known to those of skill in the field of the invention. Those skilled in the art will recognize that many of the examples provided have suitable alternatives that can be utilized. The term ‘blanket’, used to describe embodiments of the present invention, may be considered to encompass warming blankets and pads.

In order to assure maximum warming effectiveness of a patient warming blanket, a relatively high degree of accuracy in controlling the temperature in that area of the blanket which contacts the patient is required. As previously described, in order to maintain a desired heater set-point temperature, for a patient warming blanket, a controlling temperature sensor of the warming blanket should be located in a patient contact area, in order to detect the cooling of the heater caused by the heat loss to the patient. If the temperature sensor is not located at a patient contact area, the cooling of the heater, which is caused by heat being drawn from the heater, by the cool body surface of the patient, will not be detected by the sensor. A temperature sensor that does not detect the cooling will not call for more electric power, and, the areas of the heater, which are in contact with the patient, will be cooler than the set-point temperature, thereby reducing the ΔT and the resultant warming effectiveness of the blanket. For those warming blankets that include temperature sensors attached to heaters of the blankets, the temperature sensors are typically enclosed, along with the heaters, within protective outer layers of the blankets. So, without providing some indication of the location of the temperature sensors within the blankets, a clinician who is prepping a patient for surgery may not have enough information to position one of the blankets over the patient, so that the area of the blanket that corresponds to the location of the temperature sensor directly contacts the patient.

In order to address the above-described problem, some embodiments of the present invention employ a standardized location for the controlling temperature sensor(s), with respect to a visible feature of each of a particular type of warming blanket, for example, with respect to an external electrical connector of each blanket. The standardized location, once made known to clinicians, will enable the clinicians to effectively position the blankets over patients. This standardized location is that which is most likely to correspond with a patient contact area, for the particular type of blanket. However, because standardizing the location of the controlling temperature sensor(s) alone, may not reliably assure the effective positioning, every time, some embodiments of the present invention further include a visible marker to identify the location of the controlling temperature sensor(s).

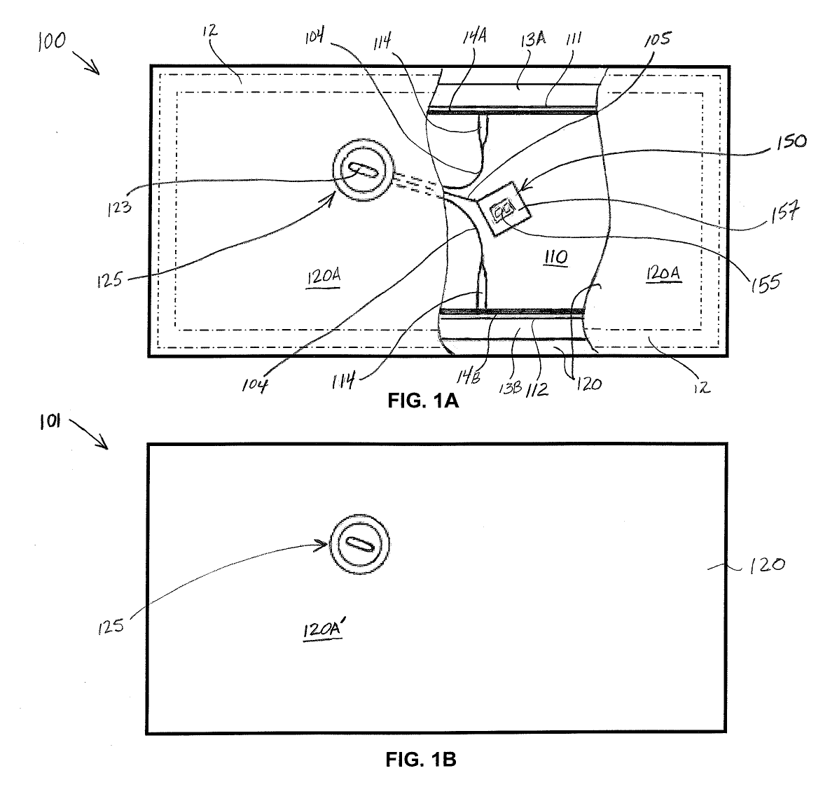

FIG. 1A is a top plan view, including a cut-away section, of a warming blanket 100, which shows a construction that may be employed by embodiments of the present invention. FIG. 1A illustrates blanket 100 including a pouch 120, which encloses a flexible heating assembly; the flexible heating assembly includes a flexible sheet-like heater 110, first and second conductive bus bars 14A, 14B, which are electrically coupled to heater 110, for powering heater 110, and a temperature sensor assembly 150, which is attached in proximity to a surface of heater 110, for providing a temperature of the heater to a temperature controller (not shown).

Flexible sheet-like heater 110 is preferably formed from a conductive fabric, which may be a carbon fiber fabric, or a woven or non-woven non-conductive substrate, for example, a woven polymer or a film polymer, into which a conductive material, for example, carbonized ink, or metalized ink, or a semi-conductive material, for example, polypyrrole, is incorporated, for example, via a printing or coating process. According to preferred embodiments, heater 110 exhibits a substantially uniform electrical resistance, across an entire surface area thereof, in order to provide a uniform watt density output, when heater 110 is electrically powered. According to an exemplary embodiment, heater 110 comprises a non-woven polyester, which has a basis weight of approximately 130 g/m² and is 100% coated with polypyrrole (available from Eeonyx Inc., Pinole, Calif.); the coated fabric has an average uniform electrical resistance, for example, determined with a four point probe measurement, of approximately 15-20 ohms per square inch at about 48 volts, which is suitable to produce a preferred uniform watt density of 0.2 to 0.4 watts/sq. in., for surface areas of heater 110 that have a width, between bus bars 14A, 14B, in the neighborhood of about 24 inches, which is suitable for a lower body heating blanket.

Sensor assembly 150 may be bonded to the surface of heater 110 with an adhesive, for example, hotmelt EVA. According to preferred embodiments, sensor assembly 150 includes a pair of temperature sensors 155, which are mounted on a heat spreader 157, for example, a copper or aluminum foil, which, preferably, has a surface area of no greater than approximately four square inches, optimally, between one and two square inches, so that sensor assembly 150 measures a relatively small area of heater 110. Pair of sensors 155 includes a primary, controlling, sensor and a secondary, redundant, sensor, which is located in close proximity to the primary sensor, as a backup to the primary sensor, for safety purposes. The mounting of the redundant sensor along with the primary sensor on the same heat spreader, and in close proximity to the primary sensor, provides assurance that the redundant sensor will measure essentially the same area of heater 110 that the primary sensor does. Each of sensors 155 may be a surface mount chip thermistor (such as a Panasonic ERT-J1VG103FA: 10K, 1% chip thermistor), and sensors 155 are preferably mounted on a substrate, for example, of polyimide (Kapton), which is bonded, for example, with a pressure sensitive adhesive, to heat spreader 157. The substrate is relatively thin, for example about 0.0005 inch thick, so that heat transfer between heat spreader 157 and sensors 155 is not significantly impeded. Other types of heat spreaders, in addition to the aforementioned metallic foils, include metallic meshes or screens, or an adhesive/epoxy filled with a thermally conductive material.

According to the illustrated embodiment, each of bus bars 14A, 14B is coupled, via a corresponding lead wire 104, to an external connector 123 of blanket 100, for connection to a power source (not shown); and sensor assembly 150 is coupled, via a lead wire 105, to external connector 123, for connection to the temperature controller of the power source. Each lead wire 104 is shown coupled to the corresponding bus bar 14A, 14B by a junction 114; and external connector 123 is shown being formed in an electrical connector housing 125, which provides a sealed electrical feedthrough, through pouch 120, from the heater assembly, enclosed within pouch 120, to an exterior of pouch 120. According to some embodiments, housing 125 is an injection molded thermoplastic, for example, PVC, and may include a flange, through which stitches can extend to secure housing 125 to pouch 120; a seal may be formed, for example, by adhesive bonding and/or heat sealing, between an inner surface of pouch 120 and a surface of the flange within pouch 120.

FIG. 1A further illustrates first bus bar 14A extending alongside a first edge 111 of heater 110, second bus bar 14B extending alongside a second edge 112 of heater 110, and first and second optional securing strips 13A, 13B extending laterally from first and second edges 111, 112, respectively. According to the illustrated embodiment, pouch 120 includes top and bottom layers which are sealed together, preferably hermetically, in a seal zone 12, which surrounds a perimeter of heater 110; seal zone 12 is shown being located to capture optional securing strips 13A, 13B so as to hold the heating assembly in a relatively fixed location within pouch 120. (Seal zone 12 is delineated with phantom lines on the top layer of pouch 120, in FIG. 1A.) It should be noted that, rather than including securing strips 13A, 13B, all or a portion of edges 111, 112 of heater 110 may extend into seal zone and include cut out areas through which opposing faces of the top and bottom layers of pouch 120 may be sealed together, in order to hold the heating assembly.

According to preferred embodiments, top and bottom layers of pouch 120 are each formed from a plastic film, which may include a fiber reinforcement and/or an anti-microbial element, for example, UltraFresh™, an antimicrobial fabric available from Thomson Research Associates. Pouch 120 protects and isolates the heating assembly from an external environment of blanket 100 and may further protect a patient disposed beneath blanket 100 from electrical shock hazards. The sealing together of top and both layers of pouch 120 may be accomplished via heat sealing.

Because temperature sensor assembly 150 is not visible from outside pouch 120, embodiments of the present invention include a visible marker, which is located on an exterior surface of pouch 120, for example, on a top exterior surface 120A, to identify a location of sensor assembly 150. The marker can aid a clinician in positioning blanket 100, over a patient, for effective warming, by locating that area of blanket 100, which corresponds to the location of sensor assembly 150, for direct contact with the patient. FIG. 1B is a plan view of an exemplary patient warming blanket 101, according to such a prior art embodiment, wherein no marker is employed. FIG. 1B illustrates blanket 101 including pouch 120 and electrical connector housing 125, like blanket 100 of FIG. 1A, and, with reference to FIG. 1B, in conjunction with FIG. 1A, it should be understood that blanket 101 further includes the same heater assembly enclosed within pouch 120. FIG. 1B further illustrates an exterior surface 120A′ of pouch 120, which is free of any visible marker to locate underlying sensor assembly 150, so that the clinician, when prepping the patient for surgery, could improperly position blanket 101 over the patient, resulting in less than effective warming of the patient.

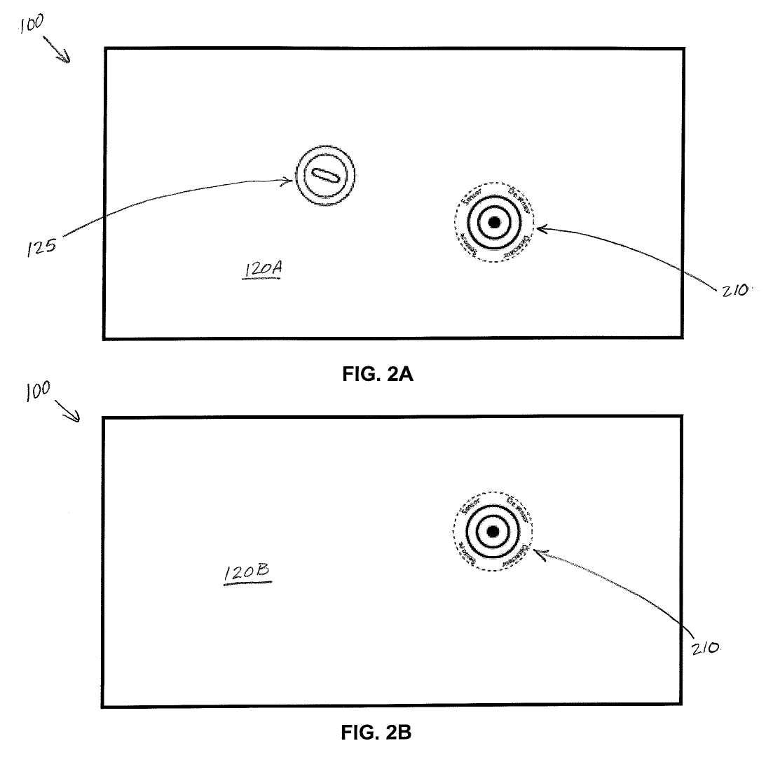

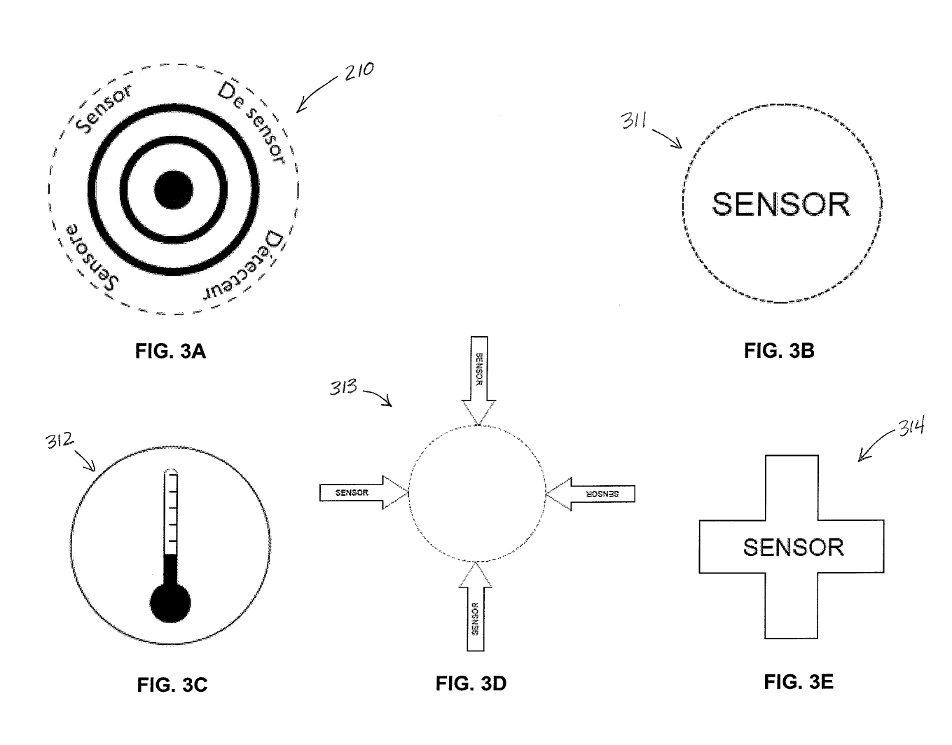

FIG. 2A is a top plan view of patient warming blanket 100, according to some embodiments of the present invention, wherein a visible marker 210 is employed. FIG. 2A illustrates marker 210 including a bulls-eye type icon and letters spelling out ‘sensor’ in various languages surrounding the icon. According to the illustrated embodiment, marker 210 is located on top exterior surface 120A of pouch 120 and is generally aligned over sensor assembly 150, which is enclosed within pouch 120. With reference to FIG. 2B, which is bottom plan view of blanket 100, according to some embodiments, marker 210 may also be located on a bottom exterior surface 120B of pouch 120, also generally aligned over sensor assembly 150. An enlarged schematic of marker 210 may be seen in FIG. 3A. FIGS. 3B-E are schematics showing alternative visible markers 311, 312, 313, 314, which may be employed by embodiments of the present invention, being located on one or both of top and bottom surfaces 120A, 120B of blanket 100, as described for marker 210.

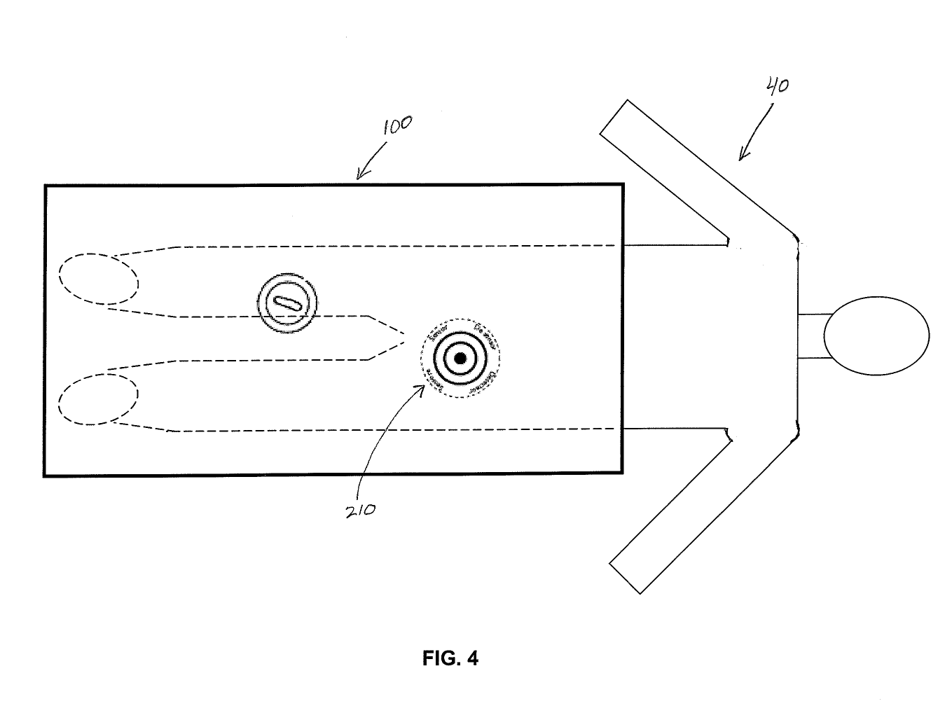

FIG. 4 is a top plan view of warming blanket 100 covering a patient 40, having been positioned according to some methods of the present invention. Portions of the patient's body beneath blanket 100 are shown with dashed lines. FIG. 4 illustrates blanket 100 having been properly positioned over patient 40, so that the area of blanket 100, that corresponds to the location of temperature sensor assembly 150 (FIG. 1A), which is identified by visible marker 210, contacts the patient's body, in order to maintain a desired set-point temperature of heater 110, as previously described. According to some methods of the present invention, the clinician, when placing blanket 100 over patient 40, will have viewed marker 210, in order to locate that area of blanket 100, which corresponds to sensor assembly 150, for direct contact with the body of patient 40.

Any of the above-described markers 210, 311, 312, 313, 314, or any other type of marker that communicates the location of temperature sensor assembly 150, may be printed on and/or embossed into one or both of exterior surfaces 120A, 120B of pouch 120, or formed on a decal/label that is applied to one or both of exterior surfaces 120A, 120B, for example, over-laid onto and adhered thereto. Alternatively, any of the aforementioned markers may be in the form of a material patch that is attached, for example, by sewing, to pouch 120.

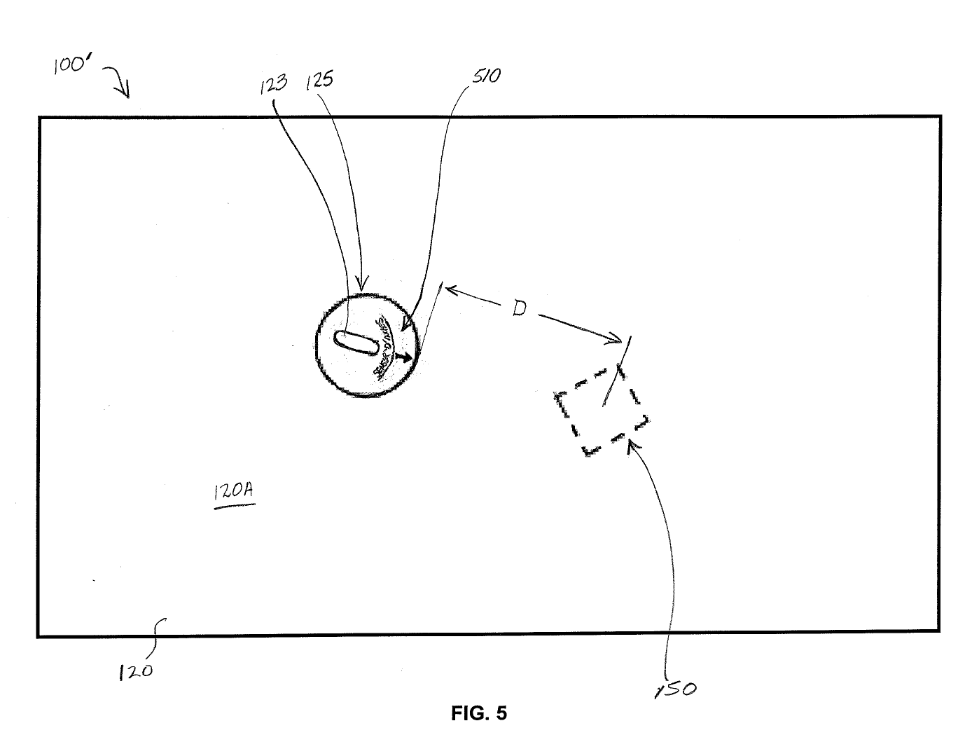

FIG. 5 is a top plan view of a warming blanket 100′, as an alternate embodiment to that of FIG. 1A. It should be noted that blanket 100′ may be similar, in many aspects, to blanket 100, and that the dashed lines in FIG. 5 denote temperature sensor assembly 150 beneath top exterior surface 120A of pouch 120. FIG. 5 illustrates blanket 100′ including a visible marker 510 that is located at an offset distance D, from sensor assembly 150, as opposed to marker 210 of blanket 100. FIG. 5 further illustrates marker 510 being located on electrical connector housing 125 of blanket 100′; marker 510 is shown including an arrow, which points toward sensor assembly 150, and words, which communicate a quantity for distance D.

Distance D may correspond to a standardized location of sensor assembly 150, with respect to connector housing 125, for every blanket of the type of blanket 100′, and, if so, the aforementioned words of marker 510 need not be necessary. Alternately, if an alignment of marker 510 and sensor assembly 150, along a predetermined axis of blanket 100′, is standardized, the arrow of marker 510 need not be necessary. Visible marker 510 may be printed on and/or embossed into a surface of connector housing 125, an integral feature of housing 125, for example, being formed during injection molding of housing 125, or formed on a decal/label that is applied to connector housing 125, for example, over-laid onto and adhered thereto. According to further embodiments, an offset visible marker, for example, similar to marker 510, may be located on one or both of exterior surfaces 120A, 120B of pouch 120, for example, in proximity to an edge thereof, being approximately aligned along a predetermined axis of blanket 100′.

In the foregoing detailed description, the invention has been described with reference to specific embodiments. However, it may be appreciated that various modifications and changes can be made without departing from the scope of the invention as set forth in the appended claims.

We claim:

1. A patient warming blanket comprising:
a flexible sheet-like heater formed from an electrically conductive fabric;
a temperature sensor assembly attached to the heater, in proximity to a surface thereof;
a pouch enclosing the heater and the temperature sensor assembly; and
a visible marker located on an exterior of the pouch, the marker identifying a location of the temperature sensor assembly enclosed within the pouch.

2. The blanket of claim 1, wherein a location of the marker is on an exterior surface of the pouch, being aligned over the location of the sensor assembly.

3. The blanket of claim 1, wherein a location of the marker is offset from the sensor assembly by a pre-determined distance, and the marker includes an arrow pointing toward the sensor assembly.

4. The blanket of claim 1, wherein a location of the marker is offset from the sensor assembly by a pre-determined distance, and the marker informs of the pre-determined distance.

5. The blanket of claim 1, further comprising:
an electrical connector housing to provide a sealed electrical feedthrough, through the pouch, from the heater and the sensor assembly to the exterior of the pouch; and
wherein a location of the marker is on the connector housing.

6. The blanket of claim 1, wherein the temperature sensor assembly comprises a heat spreader, a primary temperature sensor and secondary temperature sensor, the primary and secondary sensors being mounted on the heat spreader.

7. The blanket of claim 6, wherein the heat spreader has a surface area between approximately one square inch and approximately four square inches.

8. The blanket of claim 1, wherein the flexible sheet-like heater exhibits a uniform electrical resistance, across an entire surface area thereof, in order to provide a uniform watt density output, when the heater is electrically powered.

9. The blanket of claim 1, wherein the fabric comprises one of: a woven polymer, a non-woven polymer, and a film polymer.

10. The blanket of claim 1, wherein the fabric comprises a conductive material.

11. The blanket of claim 10, wherein the conductive material comprises one or both of: carbon and metal.

12. The blanket of claim 10, wherein the conductive material comprises a printed or coated layer of the fabric.

13. The blanket of claim 1, wherein the fabric comprises an electrically semi-conductive material.

14. The blanket of claim 13, wherein the semi-conductive material comprises polypyrrole.

15. The blanket of claim 13, wherein the semi-conductive material comprises a coated layer of the fabric.

16. The blanket of claim 1, wherein the pouch is formed from a plastic film that includes a fiber reinforcement.

17. The blanket of claim 1, wherein the visible marker comprises one or both of: a printed icon and printed words.

18. The blanket of claim 1, wherein the visible marker comprises an embossed portion of the exterior surface of the pouch.

19. The blanket of claim 1, wherein the visible marker comprises one or more decals applied to the exterior surface of the pouch.

20. The blanket of claim 1, wherein the visible marker comprises a patch of material attached to the pouch.

21. A method for positioning a patient warming blanket over a patient, in order to effectively warm the patient, the blanket comprising a pouch and a flexible heating assembly enclosed within the pouch, and the method comprising:
placing the blanket over the patient;
viewing a marker located on an exterior of the pouch;
locating an area of the blanket to directly contact the patient, the area being indicated by the marker, and the area corresponding to a location of a temperature sensor of the flexible heating assembly enclosed within the pouch.

22. The method of claim 21, wherein the marker comprises one or both of: a printed icon and printed words.

23. The method of claim 21, wherein the marker comprises an embossed portion of the exterior surface of the pouch.

24. The method of claim 21, wherein the marker comprises one or more decals applied to the exterior surface of the pouch.

25. The method of claim 21, wherein the marker comprises a patch of material attached to the pouch.
